(19) United States
(12) Patent Application Publication
(10) Pub. No.: US 2008/0287771 A1
(43) Pub. Date: Nov. 20, 2008

(54) SURGICAL NAVIGATION SYSTEM WITH ELECTROSTATIC SHIELD

(75) Inventor: Peter Traneus Anderson, Andover, MA (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/924,932

(22) Filed: Oct. 26, 2007

Related U.S. Application Data
(60) Provisional application No. 60/938,608, filed on May 17, 2007.

(51) Int. Cl. A61B 6/00; A61B 5/055

(52) U.S. Cl. 600/410; 600/424

(57) ABSTRACT

A surgical navigation system is disclosed herein. The surgical navigation system includes a tracking system, a field generator operatively connected to the tracking system, a field sensor operatively connected to the tracking system, and an electrostatic shield circumscribing the field sensor. The electrostatic shield is adapted minimize capacitive coupling between the field generator and the field sensor.

### RELATED APPLICATIONS

This application claims priority to Provisional Application No. 60/938,608 filed on May 17, 2007, and is hereby incorporated herein by reference in its entirety.

### BACKGROUND OF THE INVENTION

The subject matter disclosed herein relates to a surgical navigation system with an electrostatic shield adapted to prevent or reduce capacitive coupling.

Surgical navigation systems incorporating electromagnetic (EM) tracking technology can be implemented to determine the position and orientation of a medical instrument and to convey this information to a user. The position and orientation information may, for example, be conveyed by virtually superimposing a graphic representation of the distal end of the medical instrument onto a patient image. Accordingly, the user receives visual feedback to help navigate or guide the medical instrument to a target site.

EM tracking systems generally include a field generator and a field sensor configured to operate in combination in order to obtain the position and orientation information. The field generator and the field sensor each comprise an electrical conductor, and they are separated by atmospheric gasses that can act as an electrical insulator. It is generally well known that two electrical conductors separated by an electrical insulator define a capacitor, and that the field generator and field sensor of the EM tracking system can function as a capacitor.

“Capacitive coupling” is a term of art referring to the transfer of electricity between the conductors and through the insulator of a capacitor. One problem with EM tracking systems is that the amount of capacitive coupling is difficult to predict, and the algorithms implemented to calculate position and orientation are therefore commonly predicated on a zero capacitive coupling assumption. This assumption is potentially problematic in that capacitive coupling is generally unaccounted for and can lead to imprecision in the tracking system position and orientation estimates.

### SUMMARY OF THE INVENTION

The above-mentioned shortcomings, disadvantages and problems are addressed herein which will be understood by reading and understanding the following specification.

In an embodiment, a surgical navigation system includes a tracking system, a field generator operatively connected to the tracking system, a field sensor operatively connected to the tracking system, and an electrostatic shield circumscribing the field sensor. The electrostatic shield is adapted minimize capacitive coupling between the field generator and the field sensor.

In another embodiment, a surgical navigation system includes a computer, an imaging device connected to the computer, a display connected to the computer, and a tracking system connected to the computer. The tracking system is adapted to estimate a position and/or orientation of a medical instrument. The surgical navigation system also includes a field generator connected to the tracking system, a field sensor connected to the tracking system, and an electrostatic shield completely surrounding the field sensor. The electrostatic shield is adapted minimize capacitive coupling between the field generator and the field sensor.

In yet another embodiment, a surgical navigation system includes a computer, an imaging device connected to the computer, a display connected to the computer, and a tracking system connected to the computer. The tracking system is adapted to estimate a position and/or orientation of a medical instrument. The surgical navigation system also includes a field generator connected to the tracking system, a field sensor connected to the tracking system, and a housing assembly completely surrounding the field sensor. The housing assembly includes a structural portion adapted to protect the field sensor, and an electrostatic shield applied as a coating to the structural portion. The electrostatic shield is adapted minimize capacitive coupling between the field generator and the field sensor.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a navigation system in accordance with an embodiment; and

FIG. 2 is a detailed isometric illustration of a field sensor in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

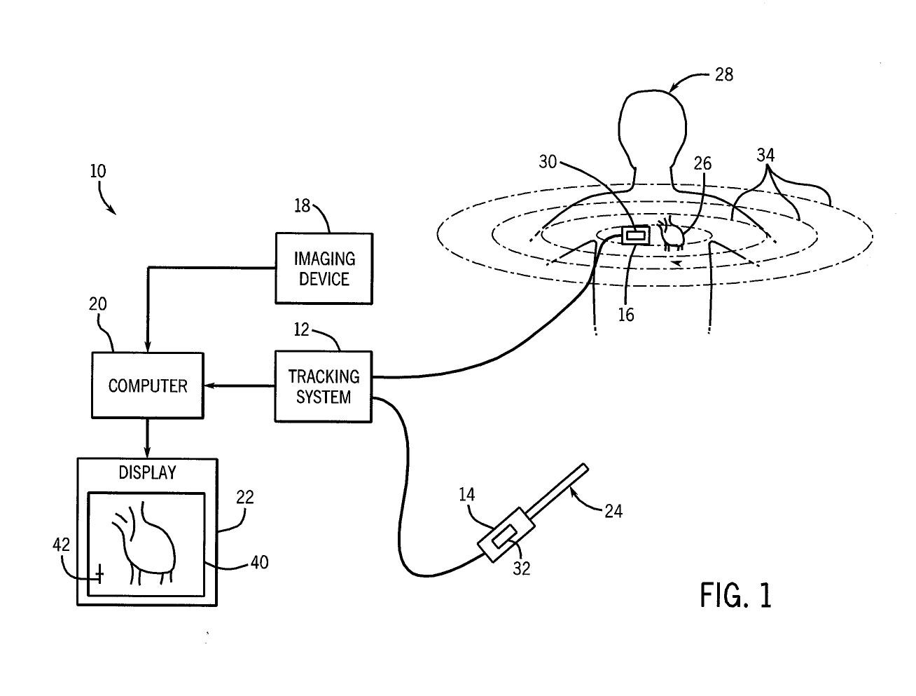

Referring to FIG. 1, a navigation system 10 is shown in accordance with one embodiment. The navigation system 10 includes an electromagnetic (EM) tracking system 12 operatively connected to a plurality of tracking elements 14, 16; an imaging device 18; a computer 20 and a display 22.

The tracking element 14 is adapted for attachment to a medical instrument such as, for example, the instrument 24. The tracking element 16 can be rigidly attached to an internal anatomy (e.g., the heart 26) or to the external body of the patient 28 in a known manner. A tracking element secured to directly to a patient may be referred to as a “dynamic reference” because it is adapted to move along with the patient. The present invention will hereinafter be described in accordance with an embodiment wherein the tracking element 16 comprises a field generator 30, and the tracking element 14 comprises a field sensor 32. It should, however, be appreciated that according to alternate embodiments the tracking element 16 may include a field sensor and the tracking element 14 may include a field generator.

The field generator 30 generates a magnetic field 34 in an area that includes the site at which a given procedure is to be performed. The field sensor 32 is adapted to measure the magnetic field 34, and to transmit the magnetic field measurements to the tracking system 12. The tracking system 12 implements the magnetic field measurements to calculate the position and orientation of the tracking element 14. After calculating the position and orientation of the tracking element 14, the position and orientation of the instrument 24 attached thereto can also be calculated in a known manner.

The tracking system 12 transmits the medical instrument position and orientation data to the computer 20. The computer 20 registers the position and orientation data to a patient image 40 obtained from the imaging device 18. The imaging device 18 may, for example, include a CT imaging device, a MR imaging device, a PET imaging device, an ultrasound imaging device, an X-ray imaging device, or any other known imaging device, as well as any combinations thereof. The medical instrument position and orientation data can be visualized on the display 22. According to one embodiment, a graphic representation corresponding to the instrument 24 can be virtually superimposed on the patient image 40 in a manner adapted to convey the position and orientation of the instrument 24. In the embodiment of FIG. 1, the graphic representation includes the cross hairs 42 which may, for example, represent the distal end portion of the instrument 24. Alternate embodiments may include a more complete rendering showing the instrument 24 in detail.

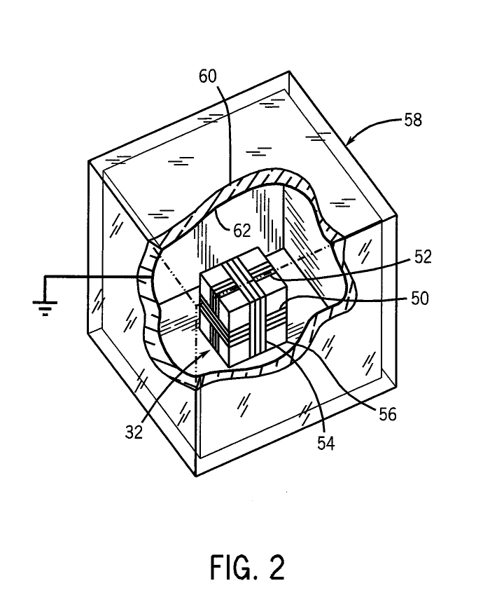

Referring to FIG. 2, a more detailed representation of the field sensor 32 is shown in accordance with an embodiment. For illustrative purposes, the field sensor 32 will hereinafter be described as comprising industry-standard-coil-architecture (ISCA) type coils, however it should be appreciated that alternate coil architectures may be envisioned. The illustrative ISCA coils of the field sensor 32 include the coils 50, 52 and 54 that are approximately collocated, approximately orthogonal, and approximately dipole coils. The coils 50, 52 and 54 may optionally be wound around a cube shaped bobbin 56 composed of an electrically insulative material such as plastic.

The coils 50, 52 and 54 may be disposed within a housing assembly 58. The housing assembly 58 comprises an electrically insulative structural portion 60 and an electrically conductive electrostatic shield 62. The structural portion 60 is adapted to protect the coils 50, 52 and 54. The electrostatic shield 62 is adapted to prevent capacitive coupling between the field generator 30 (shown in FIG. 1) and the field sensor 32, and to thereby improve the precision of the tracking system 12 (shown in FIG. 1). The electrostatic shield 62 may be grounded so that it conducts electricity in a manner that does not excessively accumulate charge. The field sensor 32 is shown disposed within the electrostatic shield 62 such that the electrostatic shield 62 completely surrounds the field sensor 32. It should, however, be appreciated that according to alternate embodiments, the electrostatic shield 62 may circumscribe only a discrete portion of the field sensor 32.

The electrostatic shield 62 may comprise any material that is conductive enough to prevent capacitive coupling and that is resistive enough to avoid the formation of eddy currents. Eddy currents can be formed by changes in the magnitude or direction of the magnetic field 34 (shown in FIG. 1) and an intersecting conductor such as the electrostatic shield. Changes in the magnitude of the magnetic field occur in the normal operation of the tracking system, and induce the voltages measured in the receiver coils. As eddy currents are a potential source of tracking system imprecision, it is important to avoid their formation by selecting a sufficiently resistive electrostatic shield material. It has been observed that materials exhibiting a resistance of approximately several ohms as measured at a distance of approximately one centimeter apart are both conductive enough to prevent capacitive coupling and resistive enough to avoid the formation of eddy currents.

According to one embodiment, the electrostatic shield 62 is applied as a coating to the internal surface of the structural portion 60. In this manner, the electrostatic shield 62 can effectively eliminate capacitive coupling by preventing the transmission of electricity between the field generator 30 (shown in FIG. 1) and the field sensor 32, and the electrostatic shield 62 is also protected by the structural portion 60. Alternatively, the electrostatic shield 62 may be applied as a coating to the external surface of the structural portion 60. The electrostatic shield 62 may, for example, be applied as a coating to the structural portion 60 by spraying an electrically conductive paint onto the structural portion 60, or by vacuum depositing an electrically conductive coating onto the structural portion 60. A non-limiting list of potentially appropriate electrostatic shield coating materials includes nickel loaded paint, silver loaded paint, carbon loaded paint, and carbon loaded plastic.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. A surgical navigation system comprising:
a tracking system;
a field generator operatively connected to the tracking system;
a field sensor operatively connected to the tracking system; and
an electrostatic shield circumscribing the field sensor, wherein the electrostatic shield is adapted minimize capacitive coupling between the field generator and the field sensor.

2. The surgical navigation system of claim 1, further comprising a structural portion of a housing assembly adapted to retain the field sensor.

3. The surgical navigation system of claim 2, wherein the electrostatic shield comprises a coating applied to the structural portion of the housing assembly.

4. The surgical navigation system of claim 2, wherein the electrostatic shield comprises a paint applied to the structural portion of the housing assembly.

5. The surgical navigation system of claim 1, wherein the electrostatic shield is sufficiently resistive to prevent the formation of eddy currents caused by the interaction of the electrostatic shield with a magnetic field produced by the field generator.

6. The surgical navigation system of claim 1, wherein the electrostatic shield comprises a material having a resistance in the range of one to ten ohms as measured at a distance of one centimeter.

7. The surgical navigation system of claim 1, wherein the field sensor is disposed within the electrostatic shield such that the electrostatic shield completely surrounds the field sensor.

8. A surgical navigation system comprising:
a computer;
an imaging device connected to the computer;
a display connected to the computer;
a tracking system connected to the computer, said tracking system adapted to estimate a position and/or orientation of a medical instrument;
a field generator connected to the tracking system;
a field sensor connected to the tracking system; and
an electrostatic shield completely surrounding the field sensor, wherein the electrostatic shield is adapted minimize capacitive coupling between the field generator and the field sensor.

9. The surgical navigation system of claim 8, wherein the imaging device comprises one of a CT imaging device; a MR imaging device; a PET imaging device; an ultrasound imaging device; and an X-ray imaging device.

10. The surgical navigation system of claim 8, wherein the display is configured to graphically convey the estimated position and/or orientation of the medical instrument.

11. The surgical navigation system of claim 8, further comprising a structural portion of a housing assembly adapted to retain the field sensor.

12. The surgical navigation system of claim 11, wherein the electrostatic shield comprises a coating applied to the structural portion of the housing assembly.

13. The surgical navigation system of claim 8, wherein the electrostatic shield is sufficiently resistive to prevent the formation of eddy currents caused by the interaction of the electrostatic shield with a magnetic field produced by the field generator.

14. A surgical navigation system comprising:
a computer;
an imaging device connected to the computer;
a display connected to the computer;
a tracking system connected to the computer, said tracking system adapted to estimate a position and/or orientation of a medical instrument;
a field generator connected to the tracking system;
a field sensor connected to the tracking system; and
a housing assembly completely surrounding the field sensor, said housing assembly comprising:
a structural portion adapted to protect the field sensor; and
an electrostatic shield applied as a coating to the structural portion of the housing assembly, wherein the electrostatic shield is adapted minimize capacitive coupling between the field generator and the field sensor.

15. The surgical navigation system of claim 14, wherein the electrostatic shield is applied as a coating to an internal surface of the structural portion.

16. The surgical navigation system of claim 14, wherein the electrostatic shield is applied as a coating to an external surface of the structural portion.

17. The surgical navigation system of claim 14, wherein the electrostatic shield comprises one of a nickel loaded paint; a silver loaded paint; and a carbon loaded paint.

18. The surgical navigation system of claim 14, wherein the imaging device comprises one of a CT imaging device; a MR imaging device; a PET imaging device; an ultrasound imaging device; and an X-ray imaging device.

19. The surgical navigation system of claim 14, wherein the display is configured to graphically convey the estimated position and/or orientation of the medical instrument.
